(19) United States
(12) Patent Application Publication
(10) Pub. No.: US 2018/0200181 A1
(43) Pub. Date: Jul. 19, 2018

(54) COMPOSITION FOR BODY SLIMMING AND SKIN CARE AND PREPARATION METHOD THEREOF

(71) Applicant: INFINITUS (CHINA) COMPANY LTD, Jiangmen, Guangdong (CN)

(72) Inventor: Xiaoying LIU, Jiangmen, Guangdong (CN)

(73) Assignee: INFINITUS (CHINA) COMPANY LTD, Jiangmen, Guangdong (CN)

(21) Appl. No.: 15/746,408

(22) PCT Filed: Mar. 16, 2016
(86) PCT No.: PCT/CN2016/076485

§ 371 Date: Jan. 20, 2018

(30) Foreign Application Priority Data
Aug. 24, 2015 (CN) 201510527307.1

(51) Int. Cl. A61K 8/9789; A61K 8/9794; A61K 8/58; A61K 8/81; A61K 8/34; A61K 8/60; A61K 8/86; A61K 8/891; A61K 8/49; A61K 8/41; A61K 8/9717; A61Q 19/00

(52) U.S. Cl. CPC A61K 8/9789; A61Q 19/00; A61K 8/585; A61K 8/8111; A61K 8/342; A61K 8/60; A61K 8/345; A61K 8/86; A61K 8/891; A61K 8/8152; A61K 8/4926; A61K 8/494; A61K 8/41; A61K 8/9717; A61K 8/9794

(57) ABSTRACT

Disclosed is a composition which has the functions of weight loss and skin care, comprising the following raw materials in parts by weight: 5-10 parts of oats, 1-3 parts of bitter orange flowers, 1-3 parts of lotus leaves and 3-5 parts of dried orange peels. The preparation method thereof is that the raw materials are pulverized and then extracted with water.

### CROSS REFERENCE TO RELATED APPLICATIONS

This application claims the priority of Chinese Patent Application No.201510527307.1 filed on Aug. 24, 2015, and the disclosures of which are hereby incorporated by reference.

### FIELD

The present invention relates to the field of biotechnology, specifically to a composition, preparation method and use thereof, and a skin care product containing the composition and preparation method thereof.

### BACKGROUND

With the increase of people's living standard, obesity has become one of the factors that affect the quality of people's life, especially for females. Stress from work and life, irregular diet, pregnancy and parturition, lack of exercise and so on become the main causes of obesity. Obesity not only affects the appearance, but also affects the body health.

At present, there are many products for eliminating obesity, including orally administered drugs and external preparations. There are two categories of commercially available weight-loss medicines: appetite suppressant that acts on the central nervous system and pancreatic lipase inhibitor. Appetite suppressant is limited in use due to its possibility of causing adverse reactions to the nervous system. Pancreatic lipase inhibitor Orlistat can help the weight loss through suppressing the decomposition and absorption of fat from foods by inhibiting the activity of pancreatic lipase. But it may cause steatorrhea and the deficiency of fat-soluble vitamin. Recently, it is also reported that Orlistat may cause damage to liver function. Although appetite suppressants lorcaserin and Qsymia have become the first weight-loss medicines authorized by FDA in 13 years, there are still some safety uncertainties about these medicines to the brain, cardiovascular system and so on. Thus, there is almost no weight-loss medicine with good efficacy and no adverse reactions on the market.

External weight-loss preparations, such as slimming cream and body shaping cream are in hot discussion. However, the efficacy of external preparations such as slimming creams on the market is not optimistic. Some products mainly rely on capsaicin to bring “hot” feeling to the skin, thereby causing consumers to mistakenly believe that the fat is burning. Other products achieve the effect of slimming through promoting the perspiration of skin and relieving edema. Due to the feature “cure the symptoms, not the disease” of these products, the weight-loss effect of these products only lasts for a short time and is easily to rebound. What is more, it may cause injury to the skin, leading to side-effect like flaccid skin, aging and so on.

Thereby, it is important to develop an external skin care preparation with effective and lasting slimming effect.

### SUMMARY

In view of above, the present disclosure provides a composition, preparation method and use thereof, and a skin care product containing the composition and preparation method thereof. The composition or skin care product is safe and non-irritating for the skin. The skin care composition and preparation obtained not only increase the secretion level of leptin and accelerate the metabolism of body fat, but also increase the content of collagen in the body, improve skin flaccid and edema caused by lipid-lowering, therefore achieving the synergetic effect of health and slimming. In addition, experiments show that the present disclosure has effect of skin repair, blood circulation improvement and anti-fatigue.

In order to achieve above objectives, the present disclosure provides technical solutions as follows:

The present disclosure provides a composition, comprising the following raw materials in parts by mass:

| | | |
|---|---|---|
| Oats | 5-10 | parts |
| Bitter orange flowers | 1-3 | parts |
| Lotus leaves | 1-3 | parts |
| Dried orange peels | 3-5 | parts |

The present disclosure also provides a method for preparing the composition, comprising: pulverizing the desired amount of raw materials according to the formula, extracting with water; the temperature for water extraction is from 61° C. to 100° C., the duration of water extraction is from 1 h to 4 h, the mass ratio of the raw materials to water is from 1:21 to 1:40.

Preferably, the temperature for water extraction in the preparation method is from 80° C. to 100° C. and the duration of water extraction is from 1 h to 4 h.

Preferably, the mass ratio of the raw materials to water in the preparation method is from 1:30 to 1:40.

Preferably, the preparation method also comprises steps of cooling, filtration and filtrate collection after water extraction.

Preferably, the filtration of the preparation method comprises common filtration and vacuum filtration.

More preferably, the common filtration is to pass 100-mesh to 200-mesh sieve.

Preferably, vacuum filtration is performed in a Buchner funnel having filter papers with 0.3 cm to 0.6 cm of diatomite in between.

Preferably, vacuum filtration is performed in a Buchner funnel having two layers of filter paper with 0.3 cm to 0.6 cm of diatomite in between.

Preferably, the filtration process is: cooling extract solution and passing it through 100-mesh sieve; performing vacuum filtration after the residue is separated out.

Preferably, the cooling in the preparation method is cooling a solution to 20° C.˜30° C.

The present disclosure also provides a composition prepared by the preparation method.

According to the “holistic, dialectical, comprehensive” theory in tradition Chinese medicine, natural botanical functional raw materials are used to increase the collagen content in the body , improve skin flaccid and edema caused by lipid-lowering, therefore achieving the aim of assisting body slimming. In addition, through ELISA method, study on the effect of the composition on leptin secreted by adipocytes shows that secretion of leptin is increased under the synergistic effect of the components. Furthermore, the components of the raw materials have effect on meridian dredging, qi-blood activating and providing qi-blood nutrition to fascia and muscle. Therefore, a slimming composition that has effect on lipometabolism regulation and anti-fatigue is obtained in view of the above.

The present disclosure also provides use of the composition and/or the composition prepared by the preparation method in the manufacture of a medicine, food, health care product and/or skin care product for slimming and skin care.

The present disclosure also provides use of the composition and/or the composition prepared by the preparation method in the manufacture of a medicine, food, health care product and/or skin care product for improving secretion of leptin by adipocyte, treating pruritus, repairing skin, removing redness and swelling, anti-fatigue, promoting blood circulation and promoting collagen synthesis.

The present disclosure also provides a skin care product, comprising the composition and/or the composition prepared by the preparation method and adjuvants acceptable in skin care products.

In some embodiments of the present disclosure, the dosage form of the skin care product can be paste, cream, essence, toner, emulsion or spray.

The present disclosure also provides a method for preparing the skin care product, which is made from the raw materials as follows:

| | | Percentage |
|---|---|---|
| Phases | INCI Name | ( wt%) |
| Phase A | Cyclotetrasiloxane | 0.5~3 wt% |
| | Cyclopentasiloxane | 0.5~3 wt% |
| | Hydrogenated Polyisobutene | 1~5 wt% |
| | Arachidyl Alcohol | 0.5~3 wt% |
| | Behenyl Alcohol | |
| | Arachidyl Glucoside | |
| | Cetearyl Alcohol | 0.01~1 wt% |
| Phase B | Water | As balance |
| | Glycerin | 1~5 wt% |
| | PEG-8 | 1~5 wt% |
| | Butanediol | 1~5 wt% |
| | Dimethicone | 0.5~3 wt% |
| | Caprylyl Methicone | 0.5~3 wt% |
| | Acrylates/C10-30 alkyl acrylate | 0.2~2 wt% |
| | crosspolymer | |
| | Niacinamide | 0.1~2 wt% |
| | Allantoin | 0.05~1 wt% |
| Phase C | Hydroxyethyl Acrylate/Sodium | 0.1~3 wt% |
| | Acryloyldimethyl Taurate | |
| | Copolymer/Squalane/Water/Polysorbate | |
| | 60/Sorbitan Isostearate | |
| Phase D | Aminomethyl Propanol | Adjust the pH |
| | | to 5.0~6.5 |
| Phase E | CafeisilaneC | 1~6 wt% |
| | Composition of claims 1 or 2 or 8 | 1~10 wt% |
| | Chondrus Crispus | 0.5~10 wt% |
| Phase F | Phenoxyethanol | 0.2~1.1 wt% |
| | Lavender Oil | 0.005~2 wt% |

The preparation method comprises steps as follows:

Step 1: heating and stirring Phase A to obtain a first product;

Step 2: heating and stirring Phase B to obtain a second product;

Step 3: mixing the first product and the second product together and emulsifying the mixture thereof to obtain a third product;

Step 4: mixing Phase C with the third product to obtain a fourth product;

Step 5: stirring and cooling the fourth product; adding Phase D, Phase E and Phase F; mixing and cooling to obtain a product.

Preferably, in the preparation method, the heating temperature in Step 1 is from 80° C. to 85° C.; the heating rate is from 1° C./min to 2° C./min; the heating duration is from 30 min to 35 min; the stirring rate in Step 1 is from 20 r/min to 60 r/min.

Preferably, in the preparation method, the heating temperature in Step 2 is from 80° C. to 85° C.; the heating rate is from 1° C./min to 2° C./min; the heating duration is from 30 min to 35 min; the stirring rate in Step 1 is from 20 r/min to 60 r/min.

Preferably, in the preparation method, the stirring rate of the mixing in Step 3 is from 30 r/min to 50 r/min; the emulsification is a homogenous emulsification processed under a condition of 2500 r/min to 3500 r/min for 3 min to 5 min.

Preferably, in the preparation method, the stirring rate of the mixing in Step 4 is from 30 r/min to 50 r/min.

Preferably, in the preparation method, the stirring rate in Step 5 is from 30 r/min to 50 r/min; the cooling temperature is from 40° C. to 50° C.; the cooling rate is from 1° C./min to 2° C./min.

Preferably, in the preparation method, the cooling temperature in Step 5 is from 30° C. to 40° C.

Preferably, the skin care product provided by the present disclosure is prepared by the method as follows:

| | | Percentage |
|---|---|---|
| Phases | INCI Name | ( wt%) |
| Phase A | Cyclotetrasiloxane | 0.5~3 wt% |
| | Cyclopentasiloxane | 0.5~3 wt% |
| | Hydrogenated Polyisobutene | 1~5 wt% |
| | Arachidyl Alcohol | 0.5~3 wt% |
| | Behenyl Alcohol | |
| | ArachidylGlucoside | |
| | Cetearyl Alcohol | 0.01~1 wt% |
| Phase B | Water | As balance |
| | Glycerin | 1~5 wt% |
| | PEG-8 | 1~5 wt% |
| | Butanediol | 1~5 wt% |
| | Dimethicone | 0.5~3 wt% |
| | Caprylyl Methicone | 0.5~3 wt% |
| | Acrylates/C10-30 alkyl | 0.2~2 wt% |
| | acrylate crosspolymer | |
| | Niacinamide | 0.1~2 wt% |
| | Allantoin | 0.05~1 wt% |
| Phase C | Hydroxy ethyl Acrylate/Sodium | 0.1~3 wt% |
| | AcryloyldimethylTaurate Copolymer/ | |
| | Squalane/Water/Polysorbate 60/Sorbitan | |
| | Isostearate | |
| Phase D | Aminomethyl Propanol | Adjust the pH |
| | | to 5.0~6.5 |
| Phase E | Cafeisilane C | 1~6 wt% |
| | Composition of claims 1 or 2 or 8 | 1~10 wt% |
| | Chondrus Crispus | 0.5~10 wt% |
| Phase F | Phenoxyethanol | 0.2~1.1 wt% |
| | Lavender Oil | 0.005~2 wt% |

(1) Phase A is added into an oil phase pot and heated to 80° C.˜85° C. with stirring. The stirring rate is from 20 r/min to 60 r/min and the heating rate is from 1° C./min to 2° C./min. The temperature is maintained constantly for 30 min to 35 min until Phase A is dissolved absolutely.

(2) Phase B is added into an aqueous phase pot and heated to 80° C.˜85° C. with stirring (the stirring rate is from 20 r/min to 60 r/min; the heating rate is from 1° C./min to 2° C./min). The temperature is maintained constantly for 30 min to 35 min.

(3) Emulsification: under condition of stirring (the stirring rate is from 30 r/min to 50 r/min), the Phase A in the oil phase pot is added into an emulsifying pot first, and then the Phase B is added into the emulsifying pot. Homogenous emulsification is performed under a condition of 2500 r/min to 3500 r/min for 3 min to 5 min.

(4) Phase C is added into the emulsifying pot under stirring, and the stirring rate is from 30 r/min to 50 r/min.

(5) Under stirring with a stirring rate of 30 r/min to 50 r/min, the temperature is cooled to 40° C.˜50° C. with a cooling rate from 1° C./min to 2° C./min; Phase D, E and F are added in turns and stirred until evenly.

(6) Mixture is stirred and cooled to below 40° C.; a product is obtained through discharge.

The present disclosure also provides a skin care product prepared by the preparation method.

In some embodiments of the present disclosure, a slimming cream is prepared by the preparation method. Using the composition in the present disclosure and the ordinary techniques and adjuvants in skin care product field, other dosage form such as essence, toner, emulsion, spray and so on can also be prepared.

Experiments show that, the composition and/or the composition prepared by the method provided by the present disclosure has effects of improving secretion of leptin by adipocyte, treating pruritus, repairing skin, removing redness and swelling, anti-fatigue, promoting blood circulation and promoting collagen synthesis.

In conclusion, the composition and/or the composition prepared by the method provided by the present disclosure have functions of slimming and skin care.

The recommended method for using the skin care product provided by the present invention is: both the composition and the cream of the present invention can be applied on the skin surface of human body, such as shank, forearm and other part in need for slimming; gently massage until it is absorbed.

Experiments demonstrate that the composition is safe and causes no irritant to skin. The skin care composition and preparation not only increase the secretion level of leptin, accelerate the metabolism of body fat, but also improve the content of collagen in the body, improve skin flaccid and edema caused by lipid-lowering, therefore achieving the aim of healthy slimming and synergistic effect. In addition, experiments show that the present disclosure also has effect on repairing skin and promoting blood circulation, so it can achieve the effects of body slimming as well as skin improvement.

### BRIEF DESCRIPTION OF DRAWINGS

In order to describe the technical solutions in the examples of the present disclosure or in the prior art more clearly, the accompanying drawings used in description of the embodiments or the prior art will be illustrated briefly.

FIG. 1 shows the infrared thermography of shank administrated with the composition of Example 1; 1(A) shows the image before use and 1(B) shows the image after use.

FIG. 2 shows the infrared thermography of forearm administrated with the composition of Example 1; 2(A) shows the image before use and 2(B) shows the image after use.

FIG. 3 shows the infrared thermography of shank administrated with the composition of Example 4; 3(A) shows the image before use and 3(B) shows the image after use.

FIG. 4 shows the infrared thermography of shank administrated with the composition of Example 4; 4(A) shows the image before use and 4(B) shows the image after use.

### DETAILED DESCRIPTION

The present disclosure discloses a composition, preparation method and use thereof, a skin care product containing the composition and the preparation method thereof. One of ordinary skill in the art can learn from the contents herein and improve the process parameters appropriately. In particular, it shall be noted that all the similar substitutions and modifications are apparent to one of ordinary skill in the art and are to be considered within the scope of the present invention. The method and application of the present invention have been described with preferred examples. It is apparent that one of the ordinary skill in the art can make change or modify the combination to the method and application of the present invention without departing from the spirit, scope and spirit of the invention, therefore realizing and applying the techniques of the present invention.

All raw materials in the examples provided by the present disclosure are commercially available. The sources of the raw materials used in the present disclosure are shown in Table 1; the manufacturers and the brands of the equipment used in the present disclosure are shown in Table 2.

TABLE 1
Raw materials and sources

| | | Percentage | |
|---|---|---|---|
| Phases | INCI Name | (%) | Manufacturer |
| Phase | Cyclotetrasiloxane | 0.5~3% | Momentive |
| A | Cyclopentasiloxane | 0.5~3% | Performance |
| | | | Materials, |
| | | | America |
| | Hydrogenated | 1~5% | Nippon Oil |
| | Polyisobutene | | Corporation, |
| | | | Japan |
| | ArachidylAlcohol/Behenyl | 0.5~3% | SEPPIC, |
| | Alcohol/ | | France |
| | ArachidylGlucoside | | |
| | MONTANOV 202 (the | | |
| | trade name of | | |
| | ArachidylAlcohol, | | |
| | Behenyl Alcohol, | | |
| | ArachidylGlucoside) | | |
| | Cetearyl Alcohol | 0.01~1% | Corning |
| Phase | Water | To 100 | Deionized |
| B | | | water, |
| | | | laboratory |
| | | | prepared |
| | Glycerin | 1~5% | SumiAsih, |
| | | | Malaysia |
| | PEG-8 | 1~5% | DOW |
| | | | CHEMICAL |
| | Butanediol | 1~5% | CELANESS, |
| | | | America |
| | Dimethicone | 0.5~3% | Dow Corning |
| | Caprylyl Methicone | 0.5~3% | Momentive |
| | | | Performance |
| | | | Materials, |
| | | | America |
| | Acrylates/C10-30 alkyl | 0.2~2% | Lubrizol |
| | acrylate crosspolymer | | Corporation |
| | Niacinamide | 0.1~2% | DSM or |
| | | | Lonza |
| | Allantoin | 0.05~1% | Kunshan |
| | | | Shuangyou |
| | | | Daily |
| | | | Chemical Co., |
| | | | Ltd. |
| Phase | Hydroxyethyl | 0.1~3% | SEPPIC, |
| C | Acrylate/Sodium | | France |
| | Acryloyldimethyl Taurate | | |
| | Copolymer/ | | |
| | Squalane/Water/Polysorbate | | |
| | 60/Sorbitan Isostearate | | |
| Phase | Aminomethyl Propanol | Adjust the pH | DOW |
| D | | to 5.0~6.5 | CHEMICAL |
| Phase | Cafeisilane C | 1~6% | EXSYMOL, |
| E | | | France |
| | Botany Extract | 1~10% | Self-made |
| | Chondrus Crispus | 0.5~10% | BASF Co., Ltd |
| Phase | Phenoxyethanol | 0.2~1.1% | Schülke |
| F | | | International |
| | Lavender Oil | 0.005~2% | Lebermuth |
| | | | Company, |
| | | | America |

TABLE 2
Equipment and manufacturer

| Name | Type | Manufacturer |
|---|---|---|
| Homoeothermic water bath | HH•S1-M | Beijing Changan Scientific |
| | | Instrument Factory |
| Intelligent Temperature | SZCL | Gongyi City, Yuhua |
| Controlling Heating Stirrer | | Instrument Co., Ltd. |
| Water Circulation Type | SHB-III | Zhengzhou Great Wall |
| Vacuum Pump | | Scientific Industrial and Trade |
| | | Co., Ltd. |

### EXAMPLES

The present disclosure is further illustrated in combination with the examples below.

### Example 1

### Preparation of the Botanical Composition

(1) The following raw materials were pulverized and then weighed according to the proportions, followed by mixing evenly.

50 g of oats, 20 g of bitter orange flowers, 20 g of lotus leaves, 50 g of dried orange peels

(2) Water was added and extraction was carried out. The mass ratio of the raw material to water is 1:21. Extraction was carried out at 61° C. for 3 h.

(3) The extract solution obtained in Step (2) was cooled to 20° C., filtered through 100-mesh sieve, and subjected to vacuum filtration after the residue was separated out. The extract solution was collected to obtain the composition.

In the vacuum filtration of Step (3), two layers of filter papers were paved in the Buchner funnel with 0.3 cm to 0.6 cm of diatomite in between.

### Example 2

### Preparation of the Botanical Composition

(1) The following raw materials were pulverized and then weighed according to the proportions, followed by mixing evenly.

65 g of oats, 30 g of bitter orange flowers, 10 g of lotus leaves, 30 g of dried orange peels

(2) Water was added and extraction was carried out. The mass ratio of the raw material to water is 1:30. Extraction was carried out at 100° C. for 1 h.

(3) The extract solution obtained in Step (2) was cooled to 25° C., filtered through 200-mesh sieve, and subjected to vacuum filtration after the residue was separated out. The extract solution was collected to obtain the composition.

In the vacuum filtration of Step (3), two layers of filter papers were paved in the Buchner funnel with 0.3 cm to 0.6 cm of diatomite in between.

### Example 3

### Preparation of the Botanical Composition

(1) The following raw materials were pulverized and then weighed according to the proportions, followed by mixing evenly.

100 g of oats, 10 g of bitter orange flowers, 30 g of lotus leaves, 40 g of dried orange peels

(2) Water was added and extraction was carried out. The mass ratio of the raw material to water is 1:40. Extraction was carried out at 80° C. for 4 h.

(3) The extract solution obtained in Step (2) was cooled to 30° C., filtered through 100-mesh sieve, and subjected to vacuum filtration after the residue was separated out. The extract solution was collected to obtain the composition.

In the vacuum filtration of Step (3), two layers of filter papers were paved in a Buchner funnel with 0.3 cm to 0.6 cm of diatomite in between.

### Example 4

### Preparation of the Slimming Cream

### Formula and Dosage

TABLE 3
Formula and dosage

| | | Percentage |
|---|---|---|
| Phases | INCI Name, herein “/” means “and” | (%) |
| Phase A | Cyclotetrasiloxane | 0.5% |
| | Cyclopentasiloxane | 0.5% |
| | Hydrogenated Polyisobutene | 2.5% |
| | Arachidyl Alcohol/Behenyl | 3% |
| | Alcohol/ArachidylGlucoside | |
| | MONTANOV 202 (trade name | |
| | of Arachidyl Alcohol, Behenyl | |
| | Alcohol, Arachidyl Glucoside) | |
| | Cetearyl Alcohol | 0.5% |
| Phase B | Water | As balance |
| | Glycerin | 3% |
| | PEG-8 | 1% |
| | Butanediol | 5% |
| | Dimethicone | 2% |
| | Caprylyl Methicone | 0.5% |
| | Acrylates/C10-30 alkyl | 1% |
| | acrylate crosspolymer | |
| | Niacinamide | 2% |
| | Allantoin | 0.05% |
| Phase C | Hydroxyethyl Aerylate/Sodium | 1.5% |
| | AcryloyldimethylTaurate Copolymer/ | |
| | Squalane/Water/Polysorbate 60/ | |
| | Sorbitan Isostearate | |
| Phase D | Aminomethyl Propanol | Adjust the pH |
| | | to 6.5 |
| Phase E | Cafeisilane C | 6% |
| | Botany Extract | 5% |
| | Chondrus Crispus | 5% |
| Phase F | Phenoxyethanol | 0.2% |
| | Lavender Oil | 2% |

### Preparation Method of the Cream:

(1) Phase A was added into an oil phase pot and heated to 80° C. with stirring. The stirring rate was 40 r/min and the heating rate was 1° C./min. The temperature was maintained constantly for 30 min until Phase A was dissolved absolutely.

(2) Phase B was added into an aqueous phase pot and heated to 80° C. with stirring (the stirring rate was 40 r/min; the heating rate was 1° C./min). The temperature was maintained constantly for 30 min.

(3) Emulsification: under condition of stirring (the stirring rate was 30 r/min), the Phase A in the oil phase pot was transferred into an emulsifying pot, and then the Phase B was transferred into the emulsifying pot. Homogenous emulsification was performed under a condition of 2500 r/min for 3 min.

(4) Phase C was added into the emulsifying pot under stirring, and the stirring rate was 30 r/min.

(5) Under the stirring with a stirring rate of 30 r/min, the temperature was cooled to 40° C. with a cooling rate of 1° C./min; Phase D, E and F were added in turns and stirred until evenly.

(6) Mixture was stirred and cooled to 40° C.; a product was obtained through discharge.

### Example 5

### Preparation of the Slimming Cream

### Formula and Dosage

TABLE 4
Formula and dosage

| | | Percentage |
|---|---|---|
| Phases | INCI Name, herein “/” means “and” | (%) |
| Phase A | Cyclotetrasiloxane | 1.5% |
| | Cyclopentasiloxane | 3% |
| | Hydrogenated Polyisobutene | 1% |
| | Arachidyl Alcohol/Behenyl Alcohol/ | 0.5% |
| | Arachidyl Glucoside | |
| | MONTANOV 202 (trade name of | |
| | Arachidyl Alcohol, Behenyl Alcohol, | |
| | Arachidyl Glucoside) | |
| | Cetearyl Alcohol | 1% |
| Phase B | Water | As balance |
| | Glycerin | 1% |
| | PEG-8 | 5% |
| | Butanediol | 3% |
| | Dimethicone | 3% |
| | Caprylyl Methicone | 1.5% |
| | Acrylates/C10-30 alkyl acrylate | 0.2% |
| | crosspolymer | |
| | Niacinamide | 1% |
| | Allantoin | 1% |
| Phase C | Hydroxyethyl Acrylate/Sodium | 0.1% |
| | AcryloyldimethylTaurate Copolymer/ | |
| | Squalane/Water/Polysorbate 60/ | |
| | Sorbitan Isostearate | |
| Phase D | Aminomethyl Propanol | Adjust the pH |
| | | to 6.0 |
| Phase E | Cafeisilane C | 3.5% |
| | Botany Extract | 10% |
| | Chondrus Crispus | 0.5% |
| Phase F | Phenoxyethanol | 0.5% |
| | Lavender Oil | 1% |

### Preparation Method of the Cream:

(1) Phase A was added into an oil phase pot and heated to 82° C. with stirring. The stirring rate was 20 r/min and the heating rate was 2° C./min. The temperature was maintained constantly for 35 min until Phase A was dissolved absolutely.

(2) Phase B was added into an aqueous phase pot and heated to 82° C. with stirring (the stirring rate was 20 r/min; the heating rate was 2° C./min). The temperature was maintained constantly for 35 min.

(3) Emulsification: under condition of stirring (the stirring rate was 40 r/min), the Phase A in the oil phase pot was transferred into an emulsifying pot first, and then the Phase B was transferred into the emulsifying pot. Homogenous emulsification was performed under a condition of 3000 r/min for 4 min.

(4) Phase C was added into the emulsifying pot under stirring, and the stirring rate was 40 r/min.

(5) Under the stirring with a stirring rate of 40 r/min, the temperature was cooled to 45° C. with a cooling rate of 2° C./min; Phase D, E and F were added in turns and stirred until evenly.

(6) Mixture was stirred and cooled to 35° C.; a product was obtained through discharge.

### Example 6

### Preparation of the Slimming Cream

### Formula and Dosage

TABLE 5
Formula and dosage

| | | Percentage |
|---|---|---|
| Phases | INCI Name, herein “/” means “and” | (%) |
| Phase A | Cyclotetrasiloxane | 3% |
| | Cyclopentasiloxane | 2% |
| | Hydrogenated Polyisobutene | 5% |
| | Arachidyl Alcohol/Behenyl Alcohol/ | 2% |
| | Arachidyl Glucoside | |
| | MONTANOV 202 (trade name of Arachidyl | |
| | Alcohol, Behenyl Alcohol, Arachidyl | |
| | Glucoside) | |
| | Cetearyl Alcohol | 0.01% |
| Phase B | Water | As balance |
| | Glycerin | 5% |
| | PEG-8 | 3% |
| | Butanediol | 1% |
| | Dimethicone | 0.5% |
| | Caprylyl Methicone | 3% |
| | Acrylates/C10-30 alkyl acrylate | 2% |
| | crosspolymer | |
| | Niacinamide | 0.1% |
| | Allantoin | 0.05% |
| Phase C | Hydroxyethyl Acrylate/Sodium | 3% |
| | AcryloyldimethylTaurate Copolymer/ | |
| | Squalane/Water/Polysorbate 60/Sorbitan | |
| | Isostearate | |
| Phase D | Aminomethyl Propanol | Adjust the pH |
| | | to 5.0 |
| Phase E | CafeisilaneC | 1% |
| | Botany extract | 1% |
| | Chondrus Crispus | 10% |
| Phase F | Phenoxyethanol | 1.1% |
| | Lavender Oil | 0.005% |

### Preparation Method of the Cream:

(1) Phase A was added into an oil phase pot and heated to 85° C. with stirring. The stirring rate was 60 r/min and the heating rate was 1° C./min. The temperature was maintained constantly for 32 min until Phase A was dissolved absolutely.

(2) Phase B was added into an aqueous phase pot and heated to 85° C. with stirring (the stirring rate was 60 r/min; the heating rate was 2° C./min). The temperature was maintained constantly for 32 min.

(3) Emulsification: under condition of stirring (the stirring rate was 50 r/min), the Phase A in the oil phase pot was transferred into an emulsifying pot first, and then the Phase B was transferred into the emulsifying pot. Homogenous emulsification was performed under a condition of 3500 r/min for 5 min.

(4) Phase C was added into the emulsifying pot under stirring, and the stirring rate was 50 r/min.

(5) Under the stirring with a stirring rate of 50 r/min, the temperature was cooled to 50° C. with a cooling rate of 2° C./min; Phase D, E and F were added in turns and stirred until evenly.

(6) Mixture was stirred and cooled to 30° C.; a product was obtained through discharge.

### Example 7

### Efficacy Experiment

### I. Effect of the Slimming Composition Prepared in Example 1, Example 2 and Example 3 on Collagen Synthesis of CCD-966SK Cells.

### 1.1 Experiment Materials

Human skin fibroblast CCD-966SK, Food Industry Research Institute, Taiwan; MEM cell culture medium (Minimum Essential Medium, Earle's), fetal bovine serum, non-essential amino acid and sodium pyruvate, from Invitrogen Corporation; dimethyl sulfoxide, Sigma-Aldrich; WST-1 (water-soluble tetrazole salt), Abnova Corporation; Sircol Collagen Assay kit, Biocolor Ltd.; samples from the examples.

### 1.2 Equipment

BP3100S electronic scales, Sartorius Intec; SZCL Intelligent Temperature Controlling Heating Stirrer, Gongyi City, Yuhua Instrument Co., Ltd.; Water Circulation Type Vacuum Pump, Zhengzhou Great Wall Scientific Industrial and Trade Co., Ltd.; M-1815 TC CO₂ incubator, America; FJ-2017 liquid scintillation counter, CNNC Xi'An Nuclear Instrument Factory; Sigma 4K15 platform high speed refrigerated centrifuge, Sigma-Aldrich, America; ADVANTAGE Vacuum Freeze Drying Device, Virtis, America; Multiskan GO enzyme-linked analyzer, Thermo.

### 1.3 Experiment Method

Human skin fibroblast CCD-966SK was cultured in MEM culture medium (containing 10% of FBS, 1% of penicillin-streptomycin, 1% of non-essential amino acid and 1% of sodium pyruvate) in an incubator with 5% of CO₂ at 37° C. Medium was changed every 2 to 3 days. Effect on cell proliferation: CCD-966SK cells were seeded in 96-well plate at a density of 1×10⁴ cell/well; the sample (prepared in complete MEM medium containing 10% of fetal bovine serum) was also added to reach a total volume of 100 μL per well. The content of collagen was determined by the Collagen Assay Kit (Sircol Collagen assay).

### 1.4 Experiment Results

The effect of the slimming composition obtained in Example 1, Example 2 and Example 3 on the collagen synthesis of human skin fibroblast CCD-966SK was evaluated. There was no botanical extract in control group. The experiment results were shown in Table 6. The results showed that the botanical extracts obtained in Examplel, Example 2 and Example 3 significantly promoted the synthesis of collagen in human skin fibroblast CCD-966SK (P<0.05).

TABLE 6
Results

| | Concentration of collagen |
|---|---|
| Sample | (mg/mL) |
| Control group | 0 |
| Example 1 | 0.02 |
| Example 2 | 0.03 |
| Example 3 | 0.03 |

II. Effect of the Slimming Composition Prepared in Example 1, Example 2 and Example 3 on Leptin Secretion from Adipocyte.

### 1. Preparation of Proliferation Culture Medium (1000 mL)

850 mL of deionized water was added to dry D-MEM/F-12(1:1) powder; after the powder dissolved totally, 2.44 g of NaHCO₃ and 3.15 g of HEPES were added. The final volume was adjusted to 1000 mL with water and pH was adjusted to 7.2 by 0.1N HCl and NaOH. The solution was sterilized by passing through 0.22 μm filtration membrane, divided into aliquots and stored at 4° C. for use. When it was used as proliferation culture medium, 10% of inactivated calf serum was added under sterile condition; when it was used as basal culture medium, 10% of inactivated fetal bovine serum was added under sterile condition.

### 2. Preparation of Differentiation Culture Medium I (100 mL)

Insulin (10 ug/mL), dexamethasone (1 μM) and IBMX (0.5 mM) were added to the basal culture medium. Medium was sterilized by passing through 0.22 μm filtration membrane, divided into aliquots and stored at 4° C. for use. Differentiation culture medium I was prepared by adding 10% of inactivated fetal bovine serum and penicillin-streptomycin under sterile condition.

### 3. Preparation of Differentiation Culture Medium II (100 mL)

1 mg of insulin was added to the basal culture medium to prepare 100 mL of differentiation culture medium with a final concentration of insulin at 10 μg/mL. The medium was sterilized by passing through 0.22 μm filtration membrane, divided into aliquots and stored at 4° C. for use. Differentiation culture medium II was prepared by adding 10% of inactivated fetal bovine serum under sterile condition.

### 4. Cell Culture

3T3-L1 preadipocytes were cultured in the proliferation medium containing 10% of calf serum in an incubator with 5% of CO₂ at 37° C. The medium was changed every 2 to 3 days, and subculture was carried out when the cells reached 50%-60% confluence. The induction of differentiation was performed according to typical cocktail method. The cell density was adjusted to 2×10⁵ cell/mL and seeded on 6-well plate or 24-well plate for subculture. The culture medium was changed every 2 to 3 days until the confluence of monolayer cells. The proliferation medium was changed to the basal culture medium containing 10% of fetal bovine serum for 2 days. The basal medium was replaced by the differentiation culture medium I for 48 h, followed by the induction in differentiation culture medium II. After 48 h, basal culture medium containing 10% of fetal bovine serum was used for the culture and changed every 48 h. 7 days later, more than 90% of the cells have a round shape, filling with lipid droplet, that is, mature adipocytes after differentiation.

### 5. Detect the Effect of Different Concentration of Act on Adipokine Through ELISA

Three parallel wells were provided in each group. Supernatants in cell culture well were collected on the tenth day and the content of leptin was detected according to the instruction of the kit. The control group was set. The samples were applied at the eighth day of differentiation.

The results of the effect of products prepared in Examples 1 to 3 on leptin secretion from mature adipocyte were shown in Table 7. As shown in Table 7, the products of Examples 1 to 3 promoted leptin secretion from mature adipocyte, resulting in the increase of leptin secretion.

TABLE 7
Results

| | Content of leptin |
|---|---|
| Sample | (ng) |
| Control Group | 12 |
| Example 1 | 16 ± 2 |
| Example 2 | 17 ± 1 |
| Example 3 | 18 ± 1 |

### III. Experiment of Anti-Inflammatory Effect of the Slimming Extract Prepared in Example 1 and Slimming Cream Prepared in Example 4 on Guinea Pig Pruritus Model

### 1. Experimental Materials

### 1.1 Experimental Samples:

Slimming extract prepared in Example 1, slimming cream prepared in Example 4

### 1.2 Experimental Animals

Healthy guinea pig, SPF/VAF grade, half male and half female, weight 250±10 g. Provided by Beijing Jinmuyang Experimental Animal Breeding Co., Ltd., Quality Certificate No. of the Animal: SCXK(Jing) 2010-0001. Raising conditions: Farm of Beijing Jinmuyang Experimental Animal Breeding Co., Ltd.

### 1.3 Reagents

Histamine Phosphate purchased from Sigma-Aldrich

### 2. Experimental Method

### 2.1 Experimental Dosage

The test samples were topically applied at an amount of 0.15 mL/cm², 0.1 mL/cm² and 0.05 mL/cm² as high dose group, medium dose group and low dose group, respectively. The model control group was topically applied with distilled water at an amount of 0.1 mL/cm².

### 2.2 Histamine Phosphate Itching Method:

36 guinea pigs were divided into groups randomly: model control group, high dose group, medium dose group and low dose group. The fur on the back of the right hind foot was shaved the day before the experiment. The samples were applied respectively on the shaved skin evenly for 3 continuous days, while the model control group was applied with distilled water. On the third day of experiment, appropriate amount of histamine phosphate was accurately weighed and diluted in distilled water to gradual concentrations of 0.01%, 0.02%, 0.03%, 0.04%, 0.05%, 0.06%, 0.07%, 0.08%, 0.09% and 0.10% before use. The shaved area on the back of right hind foot of the guinea pig was abraded by coarse sand paper for an area about 1 cm². The sample was topically applied one more time. 10 min later, 0.05 mL of 0.01% histamine phosphate was dropped on the abraded area. Thereafter, 0.05 mL histamine phosphate was dropped every 3 min at a gradual concentrations of 0.01%, 0.02%, 0.03%, 0.04% . . . , until the guinea pig started licking the right hind foot. The itching threshold was set as the total amount of histamine phosphate causing the guinea pig to start licking its right hind foot. The itching thresholds of every group were calculated and the differences between the groups were measured.

### 3. Results

The results showed that after the application of histamine phosphate to cause itch, the guinea pigs showed behavior of licking their right hind feet. After the sample was applied on the itching area, the histamine phosphate itching threshold of the high dose group increased greatly, showing significant difference compared to the model control group (P<0.05). The results were shown in Table 8.

TABLE 8
Effect of the sample on histamine phosphate itching reaction
of guinea pigs (x ± s, n = 6)

| | Dosage | Itching Threshold |
|---|---|---|
| Group | (ml/cm²) | (μg) |
| Model Control Group | 0.1 | 30.83 ± 11.14 |
| Example 4 High Dose Group | 0.15 | 58.83 ± 24.17* |
| Example 1 High Dose Group | | 50.12 ± 20.13 |
| Example 4 Middle Dose Group | 0.1 | 45.67 ± 20.17 |
| Example 1 Middle Dose Group | | 41.55 ± 21.11 |
| Example 4 Low Dose Group | 0.05 | 35.50 ± 14.75 |
| Example1 Low Dose Group | | 29.97 ± 13.56 |

Note:
Compared with the model control group,
*P < 0.05.

### 4. Conclusions

The slimming extract prepared in Example 1 and the slimming cream prepared in Example 4 can effectively increase the itching threshold of the itching caused by histamine phosphate, proving its effect of relieving dermatitis and stopping pruritus.

The slimming compositions prepared in Example 2 and Example 3 and the slimming cream prepared in Example 5 and Example 6 were used to repeat the experiment. Similar results were obtained, which have no significant difference with that of the slimming extract in Example 1 and the slimming cream in Example 4 (P>0.05).

### IV. Skin Repairing Function Evaluation of the Preparations Prepared in Example 1 and Example 4

### 1. Experimental Materials

### 1.1 Experimental Samples

Slimming extract prepared in Example 1, slimming cream prepared in Example 4.

### 1.2 Experimental Animals

36 healthy guinea pigs, SPF/VAF grade, half male and half female, weight 250±10 g. Provided by Beijing Jinmuyang Experimental Animal Breeding Co., Ltd., Quality Certificate No. of the Animal: SCXK (Jing) 2010-0001. Raising conditions: Farm of Beijing Jinmuyang Experimental Animal Breeding Co., Ltd.

### 1.3 Reagents and Equipment

Acetone and ether from Beijing Chemical Works

Skin moisture analyzer

### 2. Experimental Method

### 2.1 Experimental Dosage

The test samples were topically applied at an amount of 0.15 g/cm², 0.1 g/cm² and 0.05 g/cm² as high dose group, medium dose group and low dose group, respectively. The control group was topically applied with distilled water at an amount of 0.1 mL/cm².

2.2

36 guinea pigs were divided into groups randomly: blank control group, model control group, high dose group, medium dose group and low dose group. The fur on the nape of guinea pig was shaved at an area about 2×2 cm² the day before the experiment. Except for the blank control group, 150μL of a mixture (acetone:ether=1:1) was dropped on the shaved skin in all other groups. 10 min later, the corresponding samples were applied respectively on the shaved areas, twice a day for 5 days. Distilled water was applied to the blank control group and model control group on the shaved area. On the fifth day, the moisture content of the shaved skin was tested 20 min after the application of the sample. The differences between the groups were measured and the water loss protection rate was calculated.

The effect of the sample on skin moisture content in the skin dehydration model provided experimental basis for evaluating the repairing function of the sample to the allergic skin.

Water loss protection rate(%)=(the skin moisture content of the sample group−the skin moisture content of the model group)/the skin moisture content of the blank control group×100

### 3. Results

The results showed that after dehydration of guinea pig skin, the skin dehydration rate of the model control group was 50.75%. After application of the samples, the slimming composition prepared in Example 1 and the slimming cream prepared in Example 4 greatly increased the moisture content of the guinea pig skin, showing significant difference to that of the model control group (P<0.05). The results were shown in Table 9.

TABLE 9
Repairing function for allergic skin (x ± s, n = 6)

| | | Water loss |
|---|---|---|
| | Dosage | protection rate |
| Group | (g/cm²) | (%) |
| Blank control group | 0.1 | — |
| Model control group | 0.1 | — |
| High dose group of Example 4 | 0.15 | 32.08* |
| High dose group of Example 1 | | 29.03 |
| Medium dose group of Example | 0.1 | 25.63 |
| 4 | | |
| Medium dose group of Example | | 23.53 |
| 1 | | |
| Low dose group of Example 4 | 0.05 | 18.60 |
| Low dose group of Example 4 | | 17.10 |

### 4. Conclusion

The slimming composition prepared in Example 1 and the slimming cream prepared in Example 4 have obvious skin repairing function.

The slimming preparations prepared in Example 2 and Example 3 and the slimming cream prepared in Example 5 and Example 6 were used to repeat the above experiment. Similar effects were obtained, which have no significant difference with that of the slimming exact prepared in Example 1 and the slimming cream prepared in Example 4 (P>0.05), indicating that the preparations prepared in the present disclosure have good repairing function.

### V. Efficacy Evaluation of the Preparations Prepared in Example 1 and Example 4 on Removing Redness and Swelling

### 1. Experimental Materials

### 1.1 Experimental Samples

Slimming extract prepared in Example 1, slimming cream prepared in Example 4

### 1.2 Experimental Animals

36 SD rats, SPF/VAF grade, half male and half female, weight 180±10 g. Provided by Beijing Jinmuyang Experimental Animal Breeding Co., Ltd., Quality Certificate No. of the Animal: SCXK (Jing) 2010-0001. Raising conditions: Farm of Beijing Jinmuyang Experimental Animal Breeding Co., Ltd.

### 1.3 Reagents and Equipment

Anti-DNP IgE, DNP-HAS and Evans Blue, Sigma-Aldrich Product; acetone, Beijing Chemical Works; RT-6000 micro-plate reader, Rayto Life and Analytical Sciences Co.,Ltd..

### 2. Experimental Method

### 2.1 Experimental Dosage

The test samples were topically applied at an amount of 0.15 g/cm², 0.1 g/cm² and 0.05 g/cm² as high dose group, medium dose group and low dose group, respectively. The blank control group and the model control group were topically applied with distilled water at an amount of 0.1 g/cm².

### 2.2 Skin Allergy Test

36 rats were housed in cages at room temperature with free access to food and water for 5 day's adaptive feeding. The rats were randomly divided into groups by weight: blank control group, model control group, high dose group, medium dose group and low dose group, 6 rats per group. The fur on the back was shaved. Except for the blank control group, 0.05 μg (0.5 μl) anti-DNP IgE was subcutaneously injected in 3 spots on the back of the rat. 48 h later, 100 μg (100 μl) DNP-HAS containing 4% of Evans Blue was injected through the caudal vein of the rats. 1 h before the caudal vein injection of DNP-HAS, samples were applied to 2 cm² area surrounding the injection spot of anti-DNP IgE on the back of the rat. The rats were sacrificed 30 min after intravenous injection of DNP-HAS. The blue-dyed skin was cut off and emerged in acetone-physiological saline mixed solution (1:1) for 24 h. The solution was subjected to centrifuge to separate the supernatant, and the optical density was detected by spectrophotometer at 620 nm. Evans Blue solution was used to establish the standard curve. The pigment content of the back skin of the rat and the inhibitory rate of PCA reaction were calculated. The inhibitory rate is calculated by the formula as follow:

Inhibitory rate (%)=(pigment content of model control group−pigment content of treatment group)/(pigment content of model control group)×100

### 3. Results

As shown in the results, blue-dyed skin showed up on local skin of the rat after sensitization. The pigment content of the blue-dyed rat skin was dramatically decreased after the application of the samples, demonstrating extremely significant differences or significant differences compared to the model control group (P<0.01, P<0.05). The results were shown in Table 10.

TABLE 10
Effect of the sample on PCA reaction in the rats

| | Dosage | Inhibitory rate |
|---|---|---|
| Group | (g/cm²) | (%) |
| Blank group | 0.1 | — |
| Model group | 0.1 | — |
| High dose group of Example 4 | 0.15 | 10.89* |
| High dose group of Example 1 | | 9.11 |
| Medium dose group of Example 4 | 0.1 | 8.95 |
| Medium dose group of Example 1 | | 7.01 |
| Low dose group of Example 4 | 0.05 | 6.02 |
| Low dose group of Example 1 | | 5.48 |

### 4. Conclusion

The slimming extract prepared in Example 1 and the slimming cream prepared in Example 4 greatly decreased the pigment content of the rat blue-dyed skin after sensitization, indicating that the preparations of the present disclosure have potential function of removing redness and swelling caused by exogenous stimulation.

The preparations prepared in Example 2, Example 3, Example 5 and Example 6 were used to repeat the above experiment. Similar effects were obtained, which have no significant difference with that of the slimming exact prepared in Example 1 and the slimming cream prepared in Example 4 (P>0.05).

### VI. Evaluation of Anti-Fatigue Efficacy of the Composition Prepared in Example 1 and Cream Prepared in Example 4

In recent years, chronic fatigue caused by being sedentary and excessive mental work has become one of the common problems for office workers. The main symptoms are pain and discomfort of shoulders and neck, which seriously affects people's work efficiency and life quality. The anti-fatigue efficacy of the samples prepared in the present examples was evaluated according to the following method.

Samples: the botanic composition prepared in Example land the cream prepared in Example 4.

Subject: 30 office workers who have symptom of neck pain were divided into two groups randomly, 15 per group. The composition prepared in Example 1 and the cream prepared in Example 4 were used for trial.

Application method: smear and massage the samples on soreness area such as neck twice a day for 4 weeks, in the morning and at night, respectively.

Evaluation Standard:

significant effect: disappear of the pain ; relieved symptom: relieving of the pain; no effect: no significant difference between before and after; effective: significant improvement and the relieving of symptoms.

The statistics of the results from 30 subjects were shown in Table 11.

TABLE 11
Evaluation of anti-fatigue effect of the sample

| | | | | Effective rate |
|---|---|---|---|---|
| | Significant | Relieved | No effect | (%) |
| Example 1 | 2 | 6 | 7 | 53% |
| Example 4 | 3 | 7 | 5 | 67% |

The evaluation results showed that the composition and cream prepared in the present disclosure have effects on relieving soreness and anti-fatigue. The samples prepared in Example 2, Example 3, Example 5 and Example6 were used to repeat the above experiment. Similar effects were obtain, which have no significant difference with that of the slimming exact prepared in Example 1 and the slimming cream prepared in Example 4 (P>0.05).

### VII. Evaluation of Blood Circulation-Promoting Effect of the Preparations Prepared in Example 1 and Example 4

Sample: the botanical extract obtained in Example 1, the cream obtained in Example 4.

Application method: smear the samples on shank and forearm, twice a day for 4 weeks, in the morning and at night, respectively.

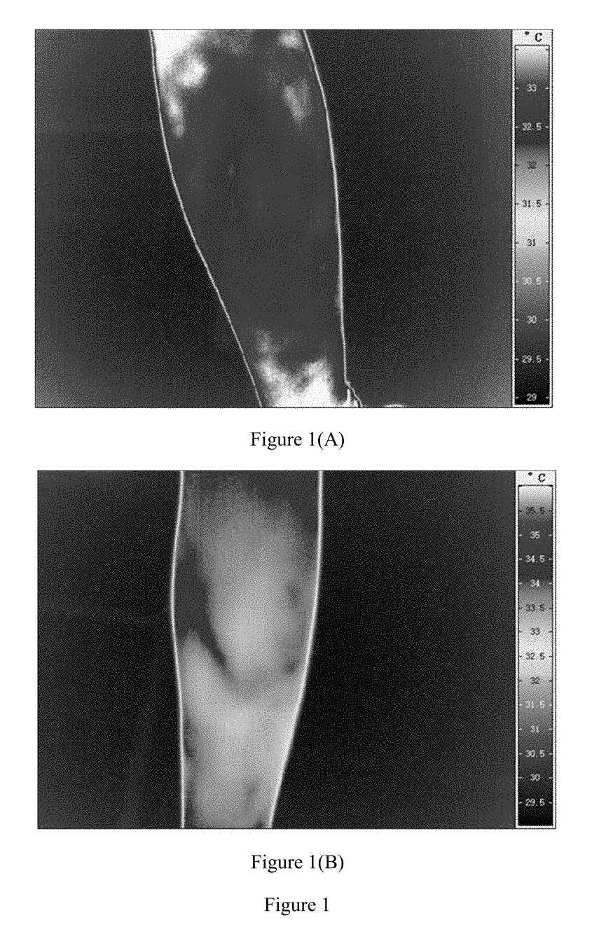

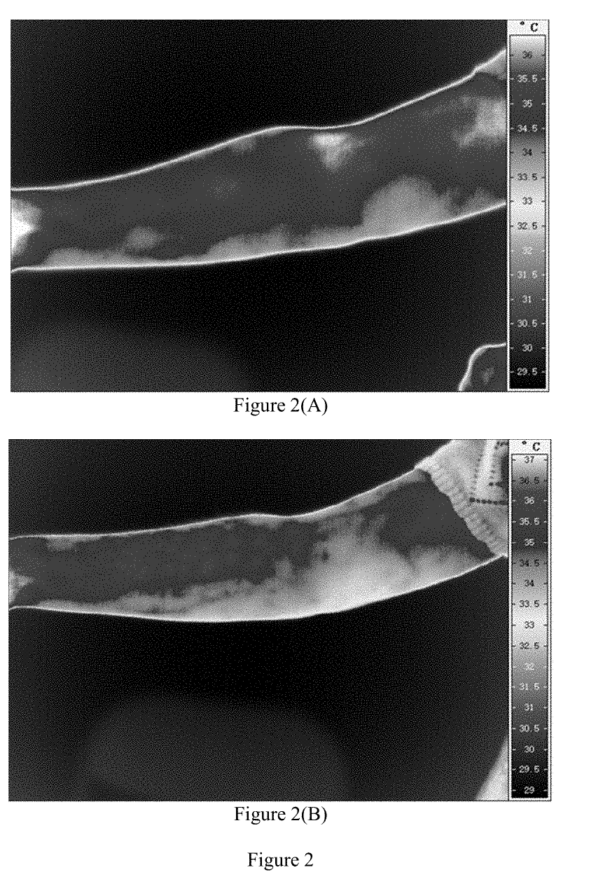

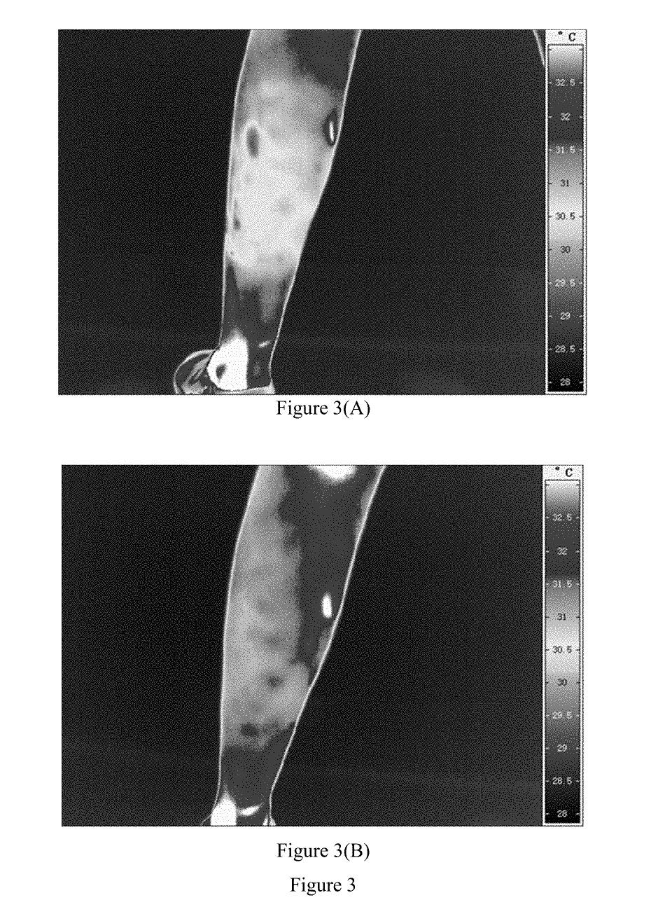

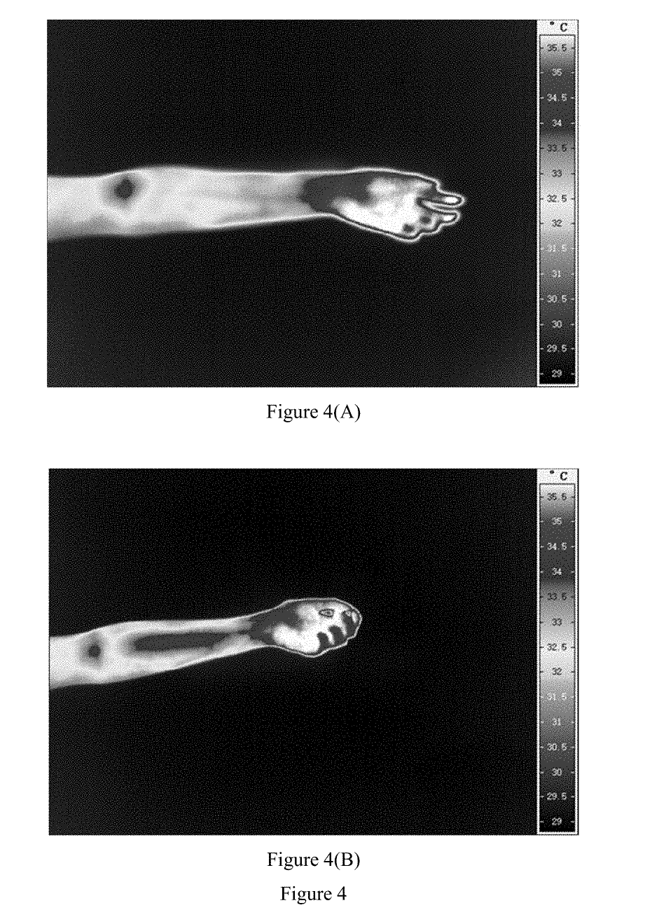

Evaluation method: the blood circulation-promoting effect of the skin care product can be estimated by infrared thermography. The infrared thermography of skin can detect the blood flow velocity and temperature of the test area, which indicates the conditions of blood circulation. Fast blood flow and high temperature means a good condition. The blood circulation-promoting effect is evaluated by comparing the infrared thermography before and after the use of samples. FIG. 1 and FIG. 2 showed the infrared thermography of shank and forearm that were applied with the composition in Example 1; FIG. 3and FIG. 4 showed the infrared thermography of shank and forearm that were applied with the composition in Example 4. As shown in the skin infrared thermography, the blood flow velocity of the subject was increased after using the samples (see FIG. 1(B), FIG. 2(B), FIG. 3(B), and FIG. 4(B)). In the figures, the test area with lighter color indicates the area with fast blood flow.

The infrared thermography demonstrates that the samples obtained in Example 1 and Example 4 have good efficacy on promoting blood circulation. The samples obtained in Example 2, Example 3, Example 5 and Example 6 were used to repeat the experiment. Similar results as above were obtained, which have no significant difference with that of the slimming extract prepared in Example 1 and the slimming cream prepared in Example 4 (P>0.05).

A composition and preparation method and use thereof, and a skin care product comprising the composition and preparation method thereof are described in detail as above. Specific examples are used to illustrate the principle and implementation manners of the present disclosure, which are merely used to help the understanding of the method and core idea of the present disclosure. It should be noted that one of ordinary skill in the art can make various improvements and modifications to the present invention without departing from the principle of the present invention, and such improvements and modifications fall into the protection scope of the claims of the present invention.

1. A composition, comprising the following raw materials in parts by mass:

| | | |
|---|---|---|
| Oats | 5 to 10 | parts |
| Bitter orange flowers | 1 to 3 | parts |
| Lotus leaves | 1 to 3 | parts |
| Dried orange peels | 3 to 5 | parts |

2. (canceled)

3. (canceled)

4. (canceled)

5. (canceled)

6. (canceled)

7. (canceled)

8. (canceled)

9. (canceled)

10. The composition according to claim 1, which is prepared by a process comprising: pulverizing the desired amount of raw materials according to the formula; extracting with water; the temperature for water extraction is from 61° C. to 100° C.; the duration of water extraction is from 1 h to 4 h; and the mass ratio of the raw materials to water is from 1:21 to 1:40.

11. The composition according to claim 1, which is a skin care product further comprising an adjuvant acceptable in skin care product.

12. The composition according to claim 11, wherein the dosage form of the skin care product is paste, cream, essence, toner, emulsion or spray.

13. A method for preparing the composition according to claim 1, comprising:
pulverizing the desired amount of raw materials according to the formula; extracting with water; the temperature for water extraction is from 61° C. to 100° C.; the duration of water extraction is from 1 h to 4 h; and the mass ratio of the raw materials to water is from 1:21 to 1:40.

14. The method according to claim 13, wherein the composition is a skin care product which is prepared by the following raw materials in parts by mass:

| | | Percentage |
|---|---|---|
| Phases | INCI Name | ( wt%) |
| Phase A | Cyclotetrasiloxane | 0.5~3 wt% |
| | Cyclopentasiloxane | 0.5~3 wt% |
| | Hydrogenated Polyisobutene | 1~5 wt% |
| | Arachidyl Alcohol | 0.5~3 wt% |
| | Behenyl Alcohol | |
| | Arachidyl Glucoside | |
| | Cetearyl Alcohol | 0.01~1 wt% |
| Phase B | Water | As balance |
| | Glycerin | 1~5 wt% |
| | PEG-8 | 1~5 wt% |
| | Butanediol | 1~5 wt% |
| | Dimethicone | 0.5~3 wt% |
| | Caprylyl Methicone | 0.5~3 wt% |
| | Acrylates/C10-30 alkyl acrylate | 0.2~2 wt% |
| | crosspolymer | |
| | Niacinamide | 0.1~2 wt% |
| | Allantoin | 0.05~1 wt% |
| Phase C | Hydroxyethyl Acrylate/Sodium | 0.1~3 wt% |
| | Acryloyldimethyl Taurate | |
| | Copolymer/Squalane/Water/ | |
| | Polysorbate-60/Sorbitan | |
| | Isostearate | |
| Phase D | Aminomethyl Propanol | Adjust the pH |
| | | to 5.0~6.5 |
| Phase E | Cafeisilane C | 1~6 wt% |
| | Composition A | 1~10 wt% |
| | Chondrus Crispus | 0.5~10 wt% |
| Phase F | Phenoxyethanol | 0.2~1.1 wt% |
| | Lavender Oil | 0.005~2 wt% |

wherein the composition A is prepared from the following raw materials in parts by mass:

| | | |
|---|---|---|
| Oats | 5 to 10 | parts, |
| Bitter orange flowers | 1 to 3 | parts, |
| Lotus leaves | 1 to3 | parts, |
| Dried orange peels | 3 to5 | parts; |

and wherein,
the preparation process comprises steps as follows:
Step 1: heating and stirring Phase A to obtain a first product;
Step 2: heating and stirring Phase B to obtain a second product;
Step 3: mixing the first product and the second product, emulsifying to obtain a third product;
Step 4: mixing Phase C with the third product to obtain a fourth product;
Step 5: stirring and cooling the fourth product, adding Phase D, Phase E and Phase F in turns, mixing and cooling to obtain a product.

15. The method according to claim 14, wherein
the heating temperature of Step 1 is from 80° C. to 85° C., the heating rate is from 1° C./min to 2° C./min, the heating duration is from 30 min to 35 min, the stirring rate in Step 1 is from 20 r/min to 60 r/min;
the heating temperature of the Step 2 is from 80° C. to 85° C., the heating rate is from 1° C./min to 2° C./min, the heating duration is from 30 min to 35 min, the stirring rate in Step 2 is from 20 r/min to 60 r/min;
the stirring rate of the mixing of Step 3 is from 30 r/min to 50 r/min; the emulsification is a homogenous emulsification under a condition of 2500 r/min to 3500 r/min for 3 min to 5 min;
the stirring rate of the mixing of Step 4 is from 30 r/min to 50 r/min;
the stirring rate of Step 5 is from 30 r/min to 50 r/min, the cooling temperature is from 40° C. to 50° C., the cooling rate is from 1° C./min to 2° C./min;
the cooling temperature of Step 5 is 30° C. to 40° C.

16. A method, comprising administrating an effective amount of the composition according to claim 1 to a human subject in need thereof.

17. The method according to claim 10, wherein the method is for slimming and skin care.

18. The method according to claim 10, wherein the method is for increasing secretion of leptin from adipocyte, treating pruritus, repairing skin, removing redness and swelling, anti-fatigue, promoting blood circulation and promoting collagen synthesis.
